(19) United States
(12) Patent Application Publication
(10) Pub. No.: US 2009/0118330 A1
(43) Pub. Date: May 7, 2009

(54) Diurea derivatives

(76) Inventors: Aina Lisbeth Abramo, Bjarred (SE); Lars Olof Goran Pettersson, Lund (SE); Kerstin Ingalill Andersson, Malmo (SE); Asa Anette Sundstedt, Malmo (SE)

Correspondence Address: BUCHANAN, INGERSOLL & ROONEY PC, POST OFFICE BOX 1404, ALEXANDRIA, VA, 22313-1404, US

(21) Appl. No.: 10/585,054

(22) PCT Filed: Jan. 19, 2005
(86) PCT No.: PCT/SE05/00054

§ 371 Date: Jul. 21, 2006

Related U.S. Application Data
(60) Provisional application No. 60/541,231, filed on Feb. 4, 2004.

(30) Foreign Application Priority Data
Feb. 4, 2004 (SE) 0400213-5

(51) Int. Cl. A61K 31/40; C07D 207/09; A61K 31/445; A61P 3/00; A61P 19/02; A61P 17/06; A61P 35/00; A61P 1/00; C07D 211/30

(52) U.S. Cl. 514/315; 548/567; 546/247; 514/428

(57) ABSTRACT

The present invention relates to novel diurea derivatives that block intracellular signal transduction and thereby inhibit the production of pro-inflammatory cytokines, especially interleukin-2 (IL-2) and/or induce apoptosis in activated T-cells. It further discloses such a compound for use as a medicament, the use of said compound for the manufacturing of a medicament for the treatment of immune disorders which benefit from inhibition of production of IL-2 and other pro-inflammatory cytokines and/or induction of apoptosis in activated T-cells, a pharmaceutical composition comprising said compound and a method of treatment comprising administration of a pharmaceutically effective amount of said compound.

### FIELD OF THE INVENTION

The present invention relates to diurea derivatives that block intracellular signal transduction and inhibit interleukin-2 (IL-2) production, to methods for their preparation, to compositions containing them and to methods and use for clinical treatment of autoimmune diseases, inflammatory diseases, organ transplant rejection and other disorders associated with IL-2 mediated immune response as well as conditions of malignant neoplasia. Because of their selective immunomodulating properties, these compounds and pharmaceutical compositions of this invention are particularly useful for preventing and treating acute or chronic inflammation, autoimmune disease (rheumatoid arthritis, multiple sclerosis, type-1 diabetes, inflammatory bowel disease, psoriasis), graft versus host disease (and other forms of organ or bone marrow transplant rejection) and malignant neoplastic disease. More particularly, the present invention relates to novel diurea derivatives suitable for the treatment of, for example, rheumatoid arthritis and graft versus host disease.

### BACKGROUND OF THE INVENTION

T lymphocytes play a central role in the immune response, both as direct effector cells and as regulatory cells that modulate the functions of numerous other cell types, primarily those that participate in the body's defence mechanisms. This regulatory function is provided either through direct cell-cell contact or via the secretion of various cytokines. Thus the proper function of T-cells is essential for the maintenance of normal homeostasis within and outside the immune system. Conversely, abnormalities in their function can lead to immunological diseases, e.g. autoimmunity, allergies and immunodeficiences. Indeed, activation of T-cells is often the initiating event in many inflammatory and autoimmune diseases.

CD4⁺ T cells of the T helper 1 (Th1) type play a pivotal role in orchestrating inflammatory immune responses. Th1 cells produce pro-inflammatory cytokines, which are commonly associated with cell-mediated immunity and induction of organ-specific autoimmune diseases (Abbas et al. 1996). The cytokine IL-2 is a principal regulator of Th1 activity (Waldmann et al. 2001). IL-2 is an autocrine growth factor that plays an essential role in the regulation of T-cell activation and proliferation. When the body launches a Th1 response against its own cells, autoimmune diseases such as rheumatoid arthritis, multiple sclerosis, type-1 diabetes, inflammatory bowel disease, and psoriasis occur. Similarly, cell-mediated immunity causes rejection of transplanted organs (allograft rejection) and graft-versus-host disease (GVHD), a serious complication that can occur after bone-marrow transplantation. In addition to IL-2, dysregulation of other pro-inflammatory Th1 cytokines (including TNF-α and IFN-γ) has also been implicated in the pathogenesis of inflammatory and autoimmune diseases (Sacca et al. 1997). Clinical studies have shown that interference with IL-2 activity effectively suppresses immune response in vivo (Waldmann et al. 1993). Accordingly, agents that inhibit IL-2 production are therapeutically useful for selectively suppressing immune response in a patient in need of such immunosuppression.

A common immunopathological hallmark of many autoimmune inflammatory diseases is a T-cell invasion and accumulation at the inflamed tissue. One mechanism implicated in this process is the failure to remove autoreactive T-cells due to defects in activation-induced cell death (Eguchi et al. 2001), suggesting that lack of apoptosis is involved in the pathogenesis of autoimmunity. Thus, approaches that attempt to correct underlying immunoregulatory defects in autoimmune disease could include inventions aimed at inhibiting cytokines (such as IL-2) and/or deleting autoreactive Th1 cells. Inappropriate survival of lymphocytes is also associated with an increased occurrence of lymphoma (Bleesing et al. 2003). Moreover, an important aspect of tumour development in general is the suppression of apoptosis, and human tumours seem to utilise several different mechanisms to evade cell suicide (White et al. 2001). Therefore, strategies to circumvent anti-apoptotic mechanisms and to activate apoptosis in tumour cells would suppress tumour formation.

In document WO03051797, small molecule inhibitors of IL-2/IL-2 receptor (IL-2R) binding are described. This approach would block the proliferative activity of IL-2/IL-2R binding but fails to inhibit other pro-inflammatory cytokines. In addition, the use of antibodies directed against IL-2Rx has been described. However, these antibodies are not orally bioavailable. Inhibition of IL-2 action can also be achieved by the use of more general immunosuppressive drugs, such as glucocorticoids, cyclosporine, azathioprione, or mycophenolate mofetil. These compounds are relatively non-selective and suffer from dose-limiting side effects. Accordingly, a need exists for compounds that effectively inhibit IL-2 production for preventing and treating immune disorders.

The prior art of IL-2 inhibition with small molecules describes no compounds structurally related to the diurea compounds of the present invention. However, other applications of related diurea derivatives have been described in the literature.

In U.S. Pat. No. 5,358,946 some urea derivatives as inhibitors of acyl-coenzyme A cholesterol acyl-transferase (ACAT) and their use for the treatment of atherosclerosis are described.

In U.S. Pat. No. 6,316,623 libraries of ethylenediamine compounds useful for screening in biological assays in order to identify pharmaceutically useful compounds are described.

In EP 0 325 397 diurea derivatives useful for the preparation of a medicament for inhibiting the acyl-coenzyme A cholesterol acyl-transferase (ACAT) enzyme in a subject are described.

In J. Am. Chem. Soc. 1995, 117, 89-99 the synthesis and conformation of 1,2-diaminoethane and 1,3-diaminopropane diureas are described.

Diurea derivatives can also be found in the CA Chemcat database. No pharmacological activities have been ascribed to these compounds.

The substitution pattern and use of the above, specifically mentioned, diureas places them outside the scope of the present invention.

### SUMMARY OF THE INVENTION

The compounds of this invention inhibit production of IL-2 and other pro-inflammatory cytokines by T-cells by inhibiting intracellular signalling. This inhibition of IL-2 is therapeutically useful for selectively suppressing immune function. Compounds also promote the induction of apoptosis in activated T-cells. The result of such selectively suppressed immunity includes reduced cell proliferation of peripheral blood lymphocytes and cellular immune response without serious toxicity or undesired side effects. Thus, the inhibition of IL-2 production and/or induction of apoptosis in activated T-cells are attractive means for preventing and treating a variety of immune disorders, including inflammatory diseases, autoimmune diseases, organ and bone marrow trans-plant rejection and other disorders associated with IL-2 mediated immune response and defective cell regulation. In particular, the compounds may be used to prevent or treat acute or chronic inflammation, rheumatoid arthritis, multiple sclerosis, type-1 diabetes, inflammatory bowel disease, psoriasis, graft versus host disease (and other forms of organ or bone marrow transplant rejection) and malignant neoplastic disease.

### DESCRIPTION OF THE DRAWING

FIG. 1. The effect (% of non-treated stimulated cells) of Compound A on PMA/Ionomycin stimulated IL-2 production in human T-cells. The curve is from a typical experiment.

### DESCRIPTION OF THE INVENTION

The objective problem of the present invention is to provide compounds which by virtue of their pharmacological profile, with high potency in experimental models and low level of side-effects, are considered to be of value in the treatment of disease associated with pathologic inflammation, autoimmunity or other pathologic cell regulation. Included in the invention is also the use of the compounds for the preparation of a medicament for the inhibition of IL-2 production. These compounds also inhibit the production of other pro-inflammatory cytokines such as tumour necrosis factor-α (TNF-α), interleukin-6 (IL-6) and interferon-γ (IFN-γ) and promote apoptosis (activation-induced cell death). In a particular aspect, this invention provides preparation of a medicament for the inhibition of IL-2 production, a method of treating diseases in which the disease pathology may be therapeutically modified by inhibiting IL-2 production and T-cell activation. Examples of such diseases are inflammatory and autoimmune diseases, organ transplant rejection, as well as malignant neoplastic diseases. In particular, the compounds may be used to prevent or treat acute or chronic inflammation, rheumatoid arthritis, multiple sclerosis, type-1 diabetes, inflammatory bowel disease, psoriasis, graft versus host disease (and other forms of organ or bone marrow transplant rejection) and malignant neoplastic disease. More particularly, the present invention relates to novel diurea derivatives-suitable for the treatment of, for example, rheumatoid arthritis and graft versus host disease.

In one aspect the present invention relates to a compound of the general formula I

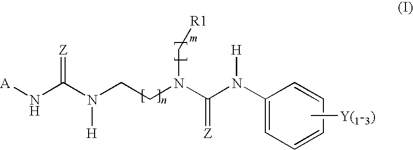

wherein

A is Ph-Y₍₁₋₃₎ or Ar—X₍₀₋₂₎;

R1 is selected from dimethylamino, diethylamino, diisopropylamino, pyrrolidino, piperidino, and 4-methyl-piperazino, and unsubstituted or substituted phenyl with substitutents selected from fluoro, chloro, bromo and methyl;

Ar is selected from phenyl, 1-naphtyl, 2-naphtyl, 2-pyridyl, 3-pyridyl, 4-pyridyl, 6-quinolinyl, and 5-pyrimidinyl;

X₍₀₋₂₎ represents 0 to 2 substituents selected from C1-C6 branched or unbranched alkyls, C1-C6 branched or unbranched alkyloxy, C1-C6 branched or unbranched acyls, fluoro, chloro, bromo, trifluoromethyl, dimethylamino, diethylamino and trifluoromethoxy;

Y₍₁₋₃₎ represents 1 to 3 substituents selected from fluoro, chloro, bromo, dimethylamino, diethylamino, trifluoromethyl, and methoxy;

Z is O or S;

n is 1-3; and

m is 1-4, preferably 2-4 or pharmaceutically acceptable salts of the compounds of the general formula I.

In one embodiment the compound have the general formula Ia

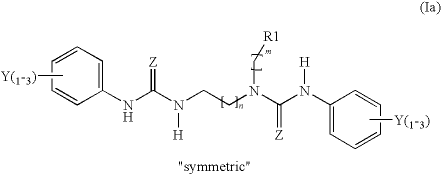

wherein

R1 is selected from dimethylamino, diethylamino, diisopropylamino, pyrrolidino, piperidino, and 4-methyl-piperazino;

Y₍₁₋₃₎ represents 1 to 3 substituents selected from fluoro, chloro, bromo, dimethylamino, diethylamino, trifluoromethyl, and methoxy;

Z is O or S;

n is 1-3; and

m is 2-4, or pharmaceutically acceptable salts of the compounds of the general formula Ia.

In another embodiment the compound have the general formula Ib

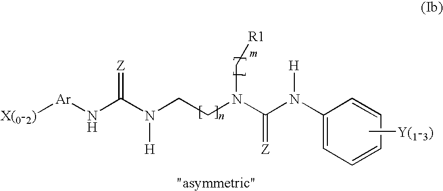

wherein

R1 is selected from dimethylamino, diethylamino, diisopropylamino, pyrrolidino, piperidino, and 4-methyl-piperazino;

Ar is selected from phenyl, 1-naphtyl, 2-naphtyl, 2-pyridyl, 3-pyridyl, 4-pyridyl, 6-quinolinyl, and 5-pyrimidinyl;

X₍₀₋₂₎ represents 0 to 2 substituents selected from C1-C6 branched or unbranched alkyls, C1-C6 branched or unbranched alkyloxy, C1-C6 branched or unbranched acyls, fluoro, chloro, bromo, trifluoromethyl, dimethylamino, diethylamino and trifluoromethoxy;

Y₍₁₋₃₎ represents 1 to 3 substituents selected from fluoro, chloro, bromo, dimethylamino, diethylamino, trifluoromethyl, and methoxy;

Z is O or S;

n is 1-3; and

m is 2-4, or pharmaceutically acceptable salts of the compounds of the general formula Ib.

In still another embodiment of the present invention

R1 is selected from dimethylamino, diethylamino, diisopropylamino, pyrrolidino, piperidino, 4-methyl-piperazino;

m is selected from 2 and 3;

n is selected from 1 and 2;

Y₍₁₋₃₎ is one substituent selected from fluoro, chloro, bromo, trifluoromethyl, dimethylamino and diethylamino.

In yet another embodiment of the present invention

Ar is selected from phenyl, 2-naphtyl and 4-pyridyl,

m is selected from 2 and 3;

Y₍₁₋₃₎ is one of the substituents selected from fluoro, chloro, bromo, and trifluoromethyl.

In another embodiment of the present invention the compound is chosen from the group comprising
1-(2-Diethylamino-ethyl)-3-(3-trifluoromethyl-phenyl)-1-{2-[3-(3-trifluoromethyl-phenyl)-ureido]-ethyl}-urea;
1-(2-Diethylamino-ethyl)-3-(4-trifluoromethyl-phenyl)-1-{2-[3-(3-trifluoromethyl-phenyl)-ureido]-ethyl}-urea;
1-(2-Pyrrolidin-1-yl-ethyl)-3-(4-trifluoromethyl-phenyl)-1-{2-[3-(4-trifluoromethyl-phenyl)-ureido]-ethyl}-urea;
3-(4-Chloro-phenyl)-1-{2-[3-(4-chloro-phenyl)-ureido]-ethyl}-1-(2-pyrrolidin-1-yl-ethyl)-urea;
1-{2-[3-(3-Chloro-phenyl)-1-(2-piperidin-1-yl-ethyl)ureido]-ethyl}-3-(3-trifluoromethyl-phenyl)-urea;
1-(2-[3-(4-Chloro-phenyl)-ureido]-ethyl}-1-(2-dimethylamino-ethyl)-3-(4-trifluoromethyl-phenyl)-urea;
3-(4-Bromo-phenyl)-1-{2-[3-(4-bromo-phenyl)-ureido]-ethyl}-1-(2-dimethylamino-ethyl)-urea;
1-(2-Diethylamino-ethyl)-1-[2-(3-phenyl-ureido)-ethyl]-3-(4-trifluoromethyl-phenyl)-urea;
1-(2-Piperidin-1-yl-ethyl)-3-(3-trifluoromethyl-phenyl)-1-{2-[3-(3-trifluoromethyl-phenyl)-ureido]-ethyl}-urea;
1-(2-Piperidin-1-yl-ethyl)-3-(4-trifluoromethyl-phenyl)-1-{2-[(3-(3-trifluoromethyl-phenyl)-ureido]-ethyl}-urea;
1-{2-[1-(2-Pyrrolidin-1-yl-ethyl)-3-(4-trifluoromethylphenyl)-ureido]-ethyl}-3-(3-trifluoromethyl-phenyl)-urea;
1-{2-[3-(4-Bromo-phenyl)-1-(2-diethylamino-ethyl)ureido]-ethyl}-3-(2,6-dichloro-pyridin-4-yl)-urea;
3-(4-Chloro-phenyl)-1-{2-[3-(4-chloro-phenyl)-ureido]-ethyl}-1-(2-diethylamino-ethyl)-urea;
1-(2-Dimethylamino-ethyl)-3-(4-trifluoromethyl-phenyl)-1-{2-[3-(3-trifluoromethyl-phenyl)-ureido]-ethyl}-urea;
1-(2-Diethylamino-ethyl)-3-(3-fluoro-phenyl)-1-{2-[3-(3-fluoro-phenyl)-ureido]-ethyl}-urea;
1-{2-[1-(3-Pyrrolidin-1-yl-propyl)-3-(4-trifluoromethylphenyl)-ureido]-ethyl}-3-(4-trifluoromethyl-phenyl)-urea;
1-{2-[3-(4-Chloro-phenyl)-ureido]-ethyl}-1-(2-diethylamino-ethyl)-3-(4-trifluoromethyl-phenyl)-urea;
1-{2-[3-(4-Chloro-phenyl)-ureido]-ethyl}-1-(2-diisopropylamino-ethyl)-3-(4-trifluoromethyl-phenyl)-urea;
1-{2-[3-(4-Chloro-phenyl)-ureido]-ethyl}-1-(2-piperidin-1-yl-ethyl)-3-(4-trifluoromethyl-phenyl)-urea;
1-(4-Chloro-phenyl)-3-{2-[3-(4-chloro-phenyl)-1-(2-diethylamino-ethyl)-thioureido]-ethyl}-thiourea;
1-{2-[3-(4-Bromo-phenyl)-ureido]-ethyl}-1-(2-diisopropylamino-ethyl)-3-(4-trifluoromethyl-phenyl)-urea;
1-(4-Chloro-phenyl)-3-{2-[1-(2-pyrrolidin-1-yl-ethyl)-3-(4-trifluoromethyl-phenyl)-ureido]-ethyl}-urea;
1-{2-[3-(4-Bromo-phenyl)-ureido]-ethyl}-1-(3-diethylamino-propyl)-3-(4-trifluoromethyl-phenyl)-urea;
1-(2-Dimethylamino-ethyl)-1-[2-(3-phenyl-ureido)-ethyl]-3-(4-trifluoromethyl-phenyl)-urea;
1-(2-Diethylamino-ethyl)-3-(4-trifluoromethyl-phenyl)-1-{2-[3-(4-trifluoromethyl-phenyl)-ureido]-ethyl}-urea;
1-(4-Bromo-phenyl)-3-{3-[1-(2-pyrrolidin-1-yl-ethyl)-3-(4-trifluoromethyl-phenyl)-thioureido]-propyl}-urea;
1-(2-Diisopropylamino-ethyl)-1-[2-(3-phenyl-ureido)ethyl]-3-(4-trifluoromethyl-phenyl)-urea;
3-(4-Chloro-phenyl)-1-(2-pyrrolidin-1-yl-ethyl)-1-{2-[3-(3-trifluoromethyl-phenyl)-ureido)-ethyl]-urea;
1-(4-Chloro-phenyl)-3-{2-[3-(3-methoxy-phenyl)-1-(2-piperidin-1-yl-ethyl)-thioureido]-ethyl}-thiourea;
3-(4-Chloro-phenyl)-1-(2-pyrrolidin-1-yl-ethyl)-1-{2-[3-(4-trifluoromethyl-phenyl)-ureido]-ethyl}-urea;
1-{2-[3-(3-Chloro-phenyl)-ureido]-ethyl}-1-(3-diethylamino-propyl)-3-(4-trifluoromethyl-phenyl)-urea; and
1-(2-Diisopropylamino-ethyl)-3-(4-trifluoromethyl-phenyl)-1-{2-[3-(4-trifluoromethyl-phenyl)-ureido]-ethyl}-urea.

In a second aspect the present invention relates to a compound as described above for use as a medicament.

In a third aspect the present invention relates to the use of a compound as described above for the manufacturing of a medicament for the treatment of immune disorders which benefit from inhibition of production of IL-2 and other pro-inflammatory cytokines and/or induction of apoptosis in activated T-cells.

In one embodiment of the use the immune disorders are chosen from the group comprising inflammatory diseases, autoimmune diseases, organ and bone marrow trans-plant rejection and other disorders associated with pro-inflammatory cytokines, especially IL-2, mediated immune response and defective cell regulation.

In another embodiment of the use the immune disorders are chosen from the group comprising acute or chronic inflammation, rheumatoid arthritis, multiple sclerosis, type-1 diabetes, inflammatory bowel disease, psoriasis, graft versus host disease and malignant neoplastic disease.

In a fourth aspect the present invention relates to a pharmaceutical composition comprising a compound as described above, admixed with one or more pharmaceutically acceptable excipients or carriers.

In one embodiment of the pharmaceutical composition the excipients are chosen from the group comprising filling agents, lubricants, flavours, colourings, sweetening, buffers, acidifying agents, diluents and preservatives.

In another embodiment the pharmaceutical composition is administered orally, intramuscularly, intravenously, intraperitoneally or subcutaneously, via implants, rectally, intranasally, transdermally, topically, or parenterally.

In a fifth aspect the present invention relates to a method of treatment comprising administration of a pharmaeutically effective amount of compound or a pharmaceutical composition as described above to a subject suffering from an immune disorder which benefit from inhibition of production of IL-2 and other pro-inflammatory cytokines and/or induction of apoptosis in autoreactive T-cells.

In one embodiment the immune disorder is chosen from the group comprising inflammatory diseases, autoimmune diseases, organ and bone marrow transplant rejection and other disorders associated with pro-inflammatory cytokines, especially IL-2, mediated immune response and defective cell regulation.

In another embodiment the immune disorders are chosen from the group comprising acute or chronic inflammation, rheumatoid arthritis, multiple sclerosis, type-1 diabetes, inflammatory bowel disease, psoriasis, graft versus host disease and malignant neoplastic disease.

All embodiments of the invention as disclosed in the claims are herewith included in the specification. The following examples are intended to illustrate the invention without restricting the scope thereof.

The compounds of general formula (I) may be prepared by methods known in the literature and the following methods.

### Method A:

The compounds of general formula (I) may be prepared by methods well known in the art. General methods of preparation are shown in Scheme A (the “symmetrical” diurea derivatives) and Scheme B (the “asymmetrical” diurea derivatives).

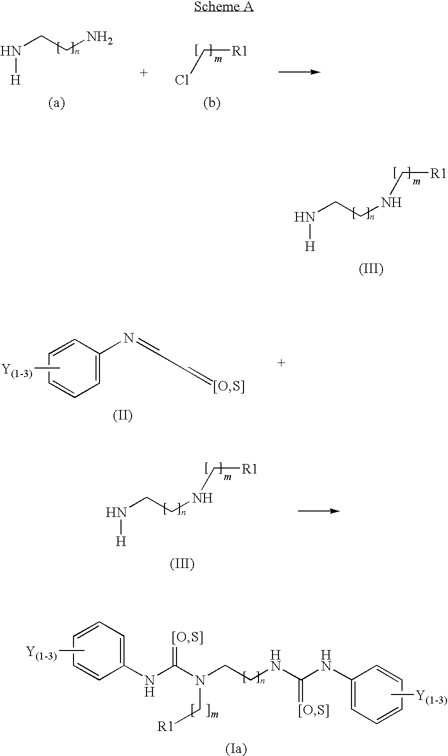

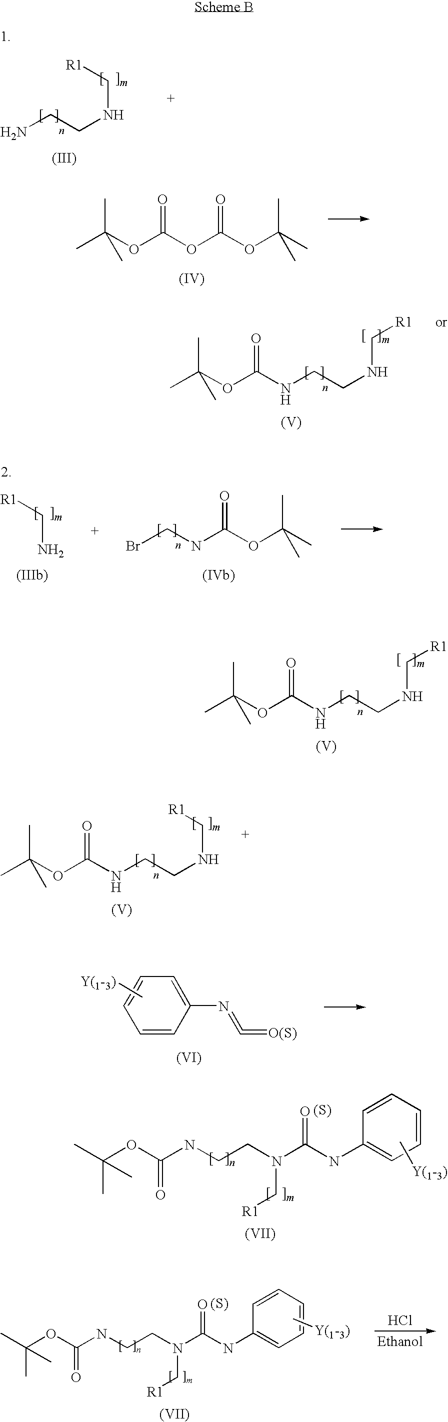

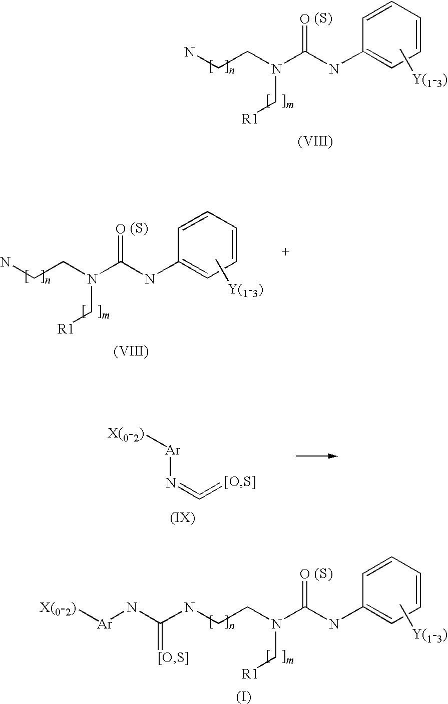

A “symmetric” diurea derivative of formula (Ia) may be prepared by conventional methods, for example, by reacting the isocyanate derivative (II) with the diamine dervative (III) in an inert solvent like dichloromethane (Scheme A). The diamine (III) may first be protected by conventional methods, like t-BOC (intermediate (V), or it can be used in excess to reduce diacylation, to produce an “asymmetric” diurea derivative (Ib) (Scheme B).

In the experimental description below AutoNom Standard was used to generate the compound names.

### Synthesis of Intermediate Derivatives (III).

### EXAMPLE 1

### N¹-(2-Pyrrolidin-1-yl-ethyl)-ethane-1,2-diamine

A solution of 1-(2-chloro-ethyl)-pyrrolidine hydrochloride (34 g, 0.2 mol) in water (20 mL) was added to a solution of ethylendiamine (24 g, 1 mol) in water (70 mL). The reaction mixture was stirred and refluxed over night. NaOH (ca 20 g) was added until the solution was saturated. The solution was extracted several times with ether. The combined organic layers was dried over potassium carbonate and evaporated. The obtained oil was distilled (108-109° C./9 mbar) to give the title compound (16 g, 51%).

Other intermediate derivatives (III), which were not commercially available, were synthesised in the same way as above.
N-(2-Amino-ethyl)-N′,N′-dimethyl-ethane-1,2-diamine b.p. 75-80° C./12-10 mbar, yield 40%.
N¹-(2-Piperidin-1-yl-ethyl)-ethane-1,2-diamine b.p. 118° C./14 mbar, yield 71%.
N-(2-Amino-ethyl)-N′,N′-dimethyl-propane-1,3-diamine b.p. 92-95° C./12-15 mbar.
N-(2-Amino-ethyl)-N′,N′-diisopropyl-ethane-1,2-diamine b.p. 110-113° C./11-14 mbar.
N-(2-Dimethylamino-ethyl)-propane-1,3-diamine b.p. 82° C./10 mbar.
N¹-(2-Pyrrolidin-1-yl-ethyl)-propane-1,3-diamine b.p. 114-120° C./11 mbar.

### Synthesis of Diurea Derivative of Formula (I)

Scheme A (“symmetric” diureas of formula Ia)

### EXAMPLE 2

### 1-(2-Pyrrolidin-1-yl-ethyl)-3-(4-trifluoromethyl-phenyl)-1-{2-[3-(4-trifluoromethyl-phenyl)-ureido]-ethyl}-urea, (Compound J)

A solution of 1-isocyanate-4-trifluoromethyl-benzene (1.05 g, 5.6 mmol) in CH₂Cl₂ (6 mL) was added dropwise to a solution of N¹— (2-pyrrolidin-1-yl-ethyl)-ethane-1,2-diamine (0.4 g, 2.5 mmol) in CH₂Cl₂ (15 mL) under N₂ at 0° C. The reaction mixture was stirred over night at room temperature. The solution was concentrated at reduced pressure and the residue was treated with CHCl₃. The title compound was precipitated and collected to give (0.9 g, 80%).

¹H NMR (CD₃OD): δ 1.83-1.90 (4H, m), 2.69 (4H, s), 2.81 (2H, t), 3.43 (2H, t), 3.55 (4H, q), 7.44-7.60 (8H, m).

Other “symmetric” diurea derivatives prepared by the method described in Example 2 are:

1-(4-Chloro-phenyl)-3-{2-[3-(4-chloro-phenyl)-1-(2-diethylamino-ethyl)-thioureido]-ethyl}-thiourea; yield 81%.

¹H NMR (CDCl₃): δ 1.03 (6H, t), 2.65 (4H, q), 2.74 (2H, d), 3.66 (2H, d), 3.95 (2H, s), 4.11 (2H, s) 7.11 (2H, d), 7.16-7.30 (4H, m), 7.35 (2H, d), 7.50 (1H, s, broad), 7.70 (1H, s, broad), 12.41 (1H, s, broad).

1-(2-Piperidin-1-yl-ethyl)-3-(3-trifluoromethyl-phenyl)-1-{2-[3-(3-trifluoromethyl-phenyl)-ureido]-ethyl}-urea, (Compound K); yield 77%.

¹H NMR (CDCl₃): δ 1.60-1.70 (6H, m), 2.50-2.67 (6H, m), 3.40-3.54 (6H, m), 6.37 (1H, s, broad), 7.14-7.26 (4H, m), 7.35 (1H, t), 7.53-7.71 (4H, m), 11.07 (1H, s, broad).

1-(2-Diethylamino-ethyl)-3-(3-trifluoromethyl-phenyl)-1-{2-[3-(3-trifluoromethyl-phenyl)-ureido]-ethyl}-urea, hydrochloride, (Compound A); yield 65%.

¹H NMR (DMSO-d₆): δ 1.25 (6H, t), 3.1-3.4 (8H, m), 3.51 (2H, t), 3.72 (2H, t), 6.9 (1H, s), 7.23 (1H, d), 7.28 (1H d), 7.42 (2H, t), 7.51 (1H, d), 7.87 (1H, d), 8.02 (2H, d) 9.14 (1H, s), 9.6 (1H, s), 9.94 (1H, s, broad).

1-(2-Diethylamino-ethyl)-3-(4-trifluoromethyl-phenyl)-1-{2-[3-(4-trifluoromethyl-phenyl)-ureido]-ethyl}-urea; yield 82%.

¹H NMR (DMSO-d₆): δ 0.99 (6H, t), 2.54-2.67 (6H, m), 3.28-3.48 (6H, m), 6.40 (H, s, broad), 7.54-7.64 (8H, m), 9.13 (1H, s).

1-(2-Diethylamino-ethyl)-3-(3-fluoro-phenyl)-1-{2-[3-(3-fluoro-phenyl)-ureido]-ethyl}-urea, hydrochloride; yield 83%.

¹H NMR (DMSO-d₆): δ 1.22 (6H, t), 3.3-3.4 (8H, m), 3.50 (2H, t) 3.68 (2H, t) 6.68-6.8 (3H, m) 7.1 (1H, d) 7.21-7.28 (2H, m) 7.38 (1H, d) 7.48 (1H, t) 7.53 (1H, d) 8.96 (1H, s) 9.34 (1H, s) 9.77 (1H, s, broad).

3-(4-Chloro-phenyl)-1-{2-[3-(4-chloro-phenyl)-ureido]-ethyl}-1-(2-diethylamino-ethyl)-urea.

¹H NMR (DMSO-d₆): δ 1.05 (6H, t), 2.58-2.73 (6H, m), 3.35-3.50 (6H, m), 6.67 (1H, s broad), 6.93 (2H, d), 7.05 (2H, d), 7.08-7.20 (4H, m), 7.67 (1H, s broad), 11.34 (1H, s, broad).

3-(4-Bromo-phenyl)-1-{2-[3-(4-bromo-phenyl)-ureido]-ethyl}-1-(2-dimethylamino-ethyl)-urea, (Compound E); yield 72%.

¹H NMR (DMSO-d₆) δ 2.25 (6H, s), 2.48 (2H, t), 3.22-3.42 (8H, m), 6.34 (1H, s), 7.36-7.42 (8H, m), 8.81 (1H, s).

3-(4-Chloro-phenyl)-1-{2-[3-(4-chloro-phenyl)-ureido]-ethyl}-1-(2-pyrrolidin-1-yl-ethyl)-urea; crystallized from CHCl₃, (Compound C); yield 71%.

¹H NMR (DMSO-d₆): δ 1.72 (4H, s), 2.54 (4H, s), 2.64 (2H, t), 3.21-3.29 (2H, t), 3.35-3.45 (4H, m), 6.31 (1H, s), 7.23-7.30 (4H, m), 7.40-7-46 (4H, m), 8.79 (1H, s), 9.67 (1H, s, broad)

1-(3-Fluoro-phenyl)-3-{2-[3-(3-fluoro-phenyl)-1-(2-pyrrolidin-1-yl-ethyl)-ureido]-ethyl}-urea; crystallized from ether; yield 85%.

¹H NMR (CDCl₃): δ 1.66 (2H, s), 1.90 (4H, s), 2.73 (4H, s), 2.83 (2H, d), 3.40-3.50 (4H, m), 6.59 (1H, s, broad), 6.61 (1H, t), 6.67 (1H, t), 6.74 (1H, d), 6.91 (1H, d), 7.00-7.20 (4H, m), 7.52 (1H, s, broad), 11.26 (1H, s, broad).

1-{2-[1-(3-Pyrrolidin-1-yl-propyl)-3-(4-trifluoromethylphenyl)-ureido]-ethyl}-3-(4-trifluoromethyl-phenyl)-urea; crystallized from CH₂Cl₂; yield 85%.

¹H NMR (CDCl₃): δ 1.81-1.89 (6H, m), 2.55-2.60 (6H, m), 3.47-3.54 (6H, m), 6.25 (1H, s, broad), 7.35 (2H, d) 7.43 (4H, d), 7.51 (2H, d), 10.32 (1H, broad).

1-(3-Dimethylamino-propyl)-3-(3-trifluoromethyl-phenyl)-1-{2-[3-(3-trifluoromethyl-phenyl)-ureido]-ethyl}-urea; yield 52%.

¹H NMR (CD₃OD): δ 1.78-1.88 (2H, m), 2.28 (6H, s), 2.39 (2H, t), 3.40-3.54 (6H, m), 7.24 (2H, s), 7.40 (2H, t), 7.53 (1H, d), 7.64 (1H, d), 7.88 (2H, d).

1-(2-Diisopropylamino-ethyl)-3-(4-trifluoromethylphenyl)-1-{2-[3-(4-trifluoromethyl-phenyl)-ureido]-ethyl}-urea; yield 48%.

¹H NMR (CD₃OD): δ 1.10 (12H, d), 2.77 (2H, s), 3.10-3.22 (2H, m), 3.43-3.54 (6H, m), 7.45-7.59 (8H, m).

1-Phenethyl-3-(4-trifluoromethyl-phenyl)-1-{2-[3-(4-trifluoromethyl-phenyl)-ureido]-ethyl}-urea; yield 91%.

¹H NMR (CDCl₃): δ 2.88 (2H, t), 3.17 (2H, s), 3.35 (2H, t), 3.53 (2H, t), 6.06 (1H, s), 7.18 (2H, d), 7.21-7.40 (6H, m), 7.41-7.49 (5H, m), 7.90 (1H, s).

### Scheme B (“Asymmetric” Diureas of Formula Ib)

Synthesis of Intermediate Derivatives (V), Alternative 1

### EXAMPLE 3

### [2-(2-Diethylamino-ethylamino)-ethyl]-carbamic Acid tert-butyl Ester

A solution of di-tert-butyl dicarbonate (690 mg, 3.1 mmol) in methanol (15 mL) was added dropwise to a stirred solution of N¹-(2-diethylamino-ethyl)-ethane-1,2-diamine (570 μl, 3.1 mmol) in methanol (15 mL) at 0° C. The reaction mixture was stirred during 3 hours and then concentrated. The crude product was purified by flash silica gel chromatography using, MeOH and MeOH/TEA 99:1 as the eluent. Concentration in vacuum of the product-rich fractions provided the title compound (728 mg, 89%).

¹H NMR (CDCl₃): δ 1.02 (6H, t), 1.46 (9H, s), 2.48-2.58 (6H, m), 2.67 (2H, t), 2.74 (2H, t), 3.17-3.29 (2H, m), 3.40-3.51 (1H, s), 5.19 (1H, s, broad).

The following intermediates type (V) were synthesised as in Example 3:
[2-(2-Pyrrolidin-1-yl-ethylamino)-ethyl]-carbamic acid tert-butyl ester.
[2-(2-Dimethylamino-ethylamino)-ethyl]-carbamic acid tert-butyl ester.
[2-(2-Piperidin-1-yl-ethylamino)-ethyl]-carbamic acid tert-butyl ester.
[2-(3-Dimethylamino-propylamino)-ethyl]-carbamic acid tert-butyl ester.
[2-(2-Diisopropylamino-ethylamino)-ethyl]-carbamic acid tert-butyl ester.
[3-(2-Dimethylamino-ethylamino)-propyl]-carbamic acid tert-butyl ester.
[3-(3-Dimethylamino-propylamino)-propyl]-carbamic acid tert-butyl ester.

Synthesis of Intermediate Derivatives (V), Alternative 2

### EXAMPLE 4

### [2-(3-Pyrrolidin-1-yl-propylamino)-ethyl]-carbamic Acid Tert-butyl Ester

(2-Bromo-ethyl)-carbamic acid tert-butyl ester (500 mg, 2.2 mmol) was added to a stirred solution of 3 pyrrolidin-1-yl-propylamine (250 mg, 2.0 mmol), NaCO₃ (504 mg, 3.6 mmol) and NaI (166 mg, 1.0 mmol) in acetonitril (30 mL). The reaction mixture was refluxed over night. Water (50 mL) was added and the mixture was extracted with CH₂Cl₂. The organic layer was dried over Na₂SO₄ and concentrated under reduced pressure to give the title compound (411 mg, 76%).

¹H NMR (CDCl₃): δ 1.43 (9H, s), 1.67 (2H, t), 1.76 (4H, s), 2.49 (4H, s), 2.55 (2H, m), 2.65 (2H, t), 2.71 (2H, t), 3.20 (2H, t).

The following intermediates type (V) were synthesised as in Example 4:
[2-(3-Diethylamino-propylamino)-ethyl]-carbamic acid tert-butyl ester.
{2-[3-(4-Methyl-piperazin-1-yl)-propylamino]-ethyl}-carbamic acid tert-butyl ester.

### Synthesis of Intermediate Derivatives (VII)

### EXAMPLE 5

### {2-[1-(2-Diethylamino-ethyl)-3-(4-trifluoromethylphenyl)-ureido]-ethyl}-carbamic Acid Tert-butyl Ester

A solution of [2-(2-diethylamino-ethylamino)-ethyl]-carbamic acid tert-butyl ester (262 mg, 1.0 mmol) and 1-isocyanate-4-trifluoromethyl-benzene (188 mg, 1.0 mmol) in CH₂Cl₂ (10 mL) was stirred for 2 hours at ambient temperature. The reaction mixture was concentrated under reduced pressure. The residue was dissolved in chloroform and passed through a Bond Elute NH₂ column. The crude product was purified by flash chromatography (SiO₂, first EtOAc and then MeOH) to give the title compound (180 mg, 40%).

¹H NMR (CDCl₃): δ 1.07 (6H, t), 1.40 (9H, s), 2.66 (6H, q) 3.30 (2H, q), 3.38 (2H, d), 3.45 (2H, t), 5.33 (1H, s), 7.41 (2H, d), 7.48 (2H, d), 11.21 (1H, s, broad).

The following intermediates type (V) were synthesised in the same way as in Example 5:
{2-[3-(4-Chloro-phenyl)-1-(2-diethylamino-ethyl)thioureido]-ethyl}-carbamic acid tert-butyl ester.
{2-[3-(4-Chloro-phenyl)-1-(2-pyrrolidin-1-yl-ethyl)ureido]-ethyl}-carbamic acid tert-butyl ester.
{2-[3-(3-Chloro-phenyl)-1-(2-piperidin-1-yl-ethyl)ureido]-ethyl}-carbamic acid tert-butyl ester.
{2-[1-(2-Dimethylamino-ethyl)-3-(4-trifluoromethylphenyl)-ureido]-ethyl}-carbamic acid tert-butyl ester.
{2-[1-(2-Pyrrolidin-1-yl-ethyl)-3-(4-trifluoromethylphenyl)-ureido]-ethyl}-carbamic acid tert-butyl ester.
{2-[1-(2-Diisopropylamino-ethyl)-3-(4-trifluoromethylphenyl)-ureido]-ethyl}-carbamic acid tert-butyl ester.
(2-[3-(4-Bromo-phenyl)-1-(2-diethylamino-ethyl)-ureido]-ethyl)-carbamic acid tert-butyl ester.
{2-[3-(4-Bromo-phenyl)-1-(2-dimethylamino-ethyl)-ureido]-ethyl}-carbamic acid tert-butyl ester.
{2-[3-(4-Bromo-phenyl)-1-(2-pyrrolidin-1-yl-ethyl)ureido]-ethyl}-carbamic acid tert-butyl ester.
{2-[3-(4-Diethylamino-phenyl)-1-(2-pyrrolidin-1-ylethyl)-thioureido]-ethyl}-carbamic acid tert-butyl ester.
{2-[1-(2-Diethylamino-ethyl)-3-(4-diethylamino-phenyl)thioureido]-ethyl}-carbamic acid tert-butyl ester.
{2-[1-[3-(4-Methyl-piperazin-1-yl)-propyl]-3-(4-trifluoromethyl-phenyl)-ureido]-ethyl}-carbamic acid tert-butyl ester.
{2-[3-(4-Methoxy-phenyl)-1-(2-pyrrolidin-1-yl-ethyl)-ureido]-ethyl}-carbamic acid tert-butyl ester.
{3-[1-(3-Dimethylamino-propyl)-3-(4-trifluoromethylphenyl)-ureido]-propyl}-carbamic acid tert-butyl ester.
{2-[1-(3-Dimethylamino-propyl)-3-(4-trifluoromethylphenyl)-ureido]-ethyl}-carbamic acid tert-butyl ester.
{2-[1-(3-Diethylamino-propyl)-3-(4-trifluoromethylphenyl)-ureido]-ethyl}-carbamic acid tert-butyl ester.

### Synthesis of Intermediate Derivatives (VIII)

### EXAMPLE 6

### 1-(2-Amino-ethyl)-1-(2-diethylamino-ethyl)-3-(4-trifluoromethyl-phenyl)-urea

[2-(2-Diethylamino-ethylamino)-ethyl]-carbamic acid tert-butyl ester (180 mg, 0.4 mmol) was suspended in a 2M solution of HCl in ethanol and stirred for 30 minutes at ambient temperature. The reaction mixture was concentrated under reduced pressure. The residue was dissolved in CHCl₃ and washed with a saturated solution of NaHCO₃. The organic layer was dried over MgSO₄ and concentrated under reduced pressure to give the crude title compound (124 mg, 89%).

¹H NMR (CDCl₃): δ 1.08 (6H, t), δ 1.43 (2H, s), 2.60-2.70 (6H, m), 2.90 (2H, t) 3.36-3.43 (4H, m), 7.40 (2H, d), 7.46 (2H, d), 11.15 (1H, s).

The other intermediates type (VIII) were synthesised in the same way as Example 6.
1-(2-Amino-ethyl)-3-(4-chloro-phenyl)-1-(2-diethylaminoethyl)-thiourea.
1-(2-Amino-ethyl)-3-(4-chloro-phenyl)-1-(2-pyrrolidin-1-yl-ethyl)-urea.
1-(2-Amino-ethyl)-3-(4-methoxy-phenyl)-1-(2-pyrrolidin-1-yl-ethyl)-urea.
1-(2-Amino-ethyl)-3-(3-chloro-phenyl)-1-(2-piperidin-1-yl-ethyl)-urea.

### Synthesis of the “Asymmetric” Diurea Derivatives of Formula (Ib)

### EXAMPLE 7

### 1-(2-Diethylamino-ethyl)-3-(4-trifluoromethyl-phenyl)-1-{2-[3-(3-trifluoromethyl-phenyl)-ureido]-ethyl}-urea, (Compound B)

A solution of 1-(2-amino-ethyl)-1-(2-diethylaminoethyl)-3-(4-trifluoromethyl-phenyl)-urea (78.5 mg, 0.23 mmol) in CH₂Cl₂ (10 mL) was stirred at ambient temperature and 1-isocyanate-3-trifluoromethyl-benzene (31.2 μl, 0.23 mmol) was added. The reaction mixture was stirred for one hour at room temperature and then concentrated under reduced pressure. The crude material was purified by flash silica gel chromatography using, MeOH as the eluent. Concentration in vacuum of the product-rich fractions provided the title compound (92 mg, 76%).

¹H NMR (CDCl₃): δ 1.10 (6H, 2t), 2.65-2.77 (6H, m), 3.45 (6H, s), 6.64 (1H, s, broad), 7.06-7.23 (3H, m), 7.35 (2 H, d) 7.46 (2H, d), 7.58 (1H, s), 7.98 (1H, s, broad), 11.70 (1H, s, broad).

Other “asymmetric” diurea derivatives of the formula (I), prepared by the method described in Example 7, are:

1-(4-Chloro-phenyl)-3-{2-[3-(4-chloro-phenyl)-1-(2-diethylamino-ethyl)-thioureido]-ethyl}-urea; yield 51%.

¹H NMR (CDCl₃): δ 1.01 (6H, t), 2.63 (4H, q), 2.72 (2H, d), 3.55-3.69 (4H, m), 4.04 (2H, t), 6.18 (1H, s, broad), 7.18 (4H, t) 7.25-7.32 (2H, m), 12.6 (1H, s, broad).

1-{2-[3-(4-Chloro-phenyl)-ureido]-ethyl}-1-(2-dimethylamino-ethyl)-3-(4-trifluoromethyl-phenyl)-urea, hydrochloride (Compound L).

¹H NMR (DMSO-d₆): δ 2.82 (6H, 2s), 3.20-3.40 (4H, m), 3.49 (2H, t), 3.71 (2H, t), 6.79 (1H, t), 7.26 (2H, d), 7.43 (2H, d), 7.58 (2H, d), 7.81 (2H, d), 9.14 (1H, s), 9.31 (1H, s), 9.91 (1H, s, broad).

1-(2-Dimethylamino-ethyl)-1-[2-(3-phenyl-ureido)-ethyl]-3-(4-trifluoromethyl-phenyl)-urea; yield 71%.

¹H NMR (CDCl₃): δ 2.37 (6H, s), 2.60 (2H, s), 3.34 (6H, s), 6.52 (1H, s), 6.91-6.99 (1H, m), 7.15 (4H, d), 7.34 (2H, d), 7.46 (2H, d), 7.71 (1H, s), 11.58 (1H, s, broad).

1-(2-Diethylamino-ethyl)-1-[2-(3-phenyl-ureido)-ethyl]-3-(4-trifluoromethyl-phenyl)-urea, (Compound F); yield 74%.

¹H NMR (CDCl₃): δ 1.06 (6H, t), 2.59-2.71 (6H, m), 3.45 (6H, s), 6.54 (1H, s), 6.89-6.97 (1H, m), 7.14 (4H, d), 7.35 (2H, d), 7.46 (2H, d), 7.77 (1H, s), 11.55 (1H, s, broad).

1-{2-[3-(4-Chloro-phenyl)-ureido]-ethyl}-1-(2-diethylamino-ethyl)-3-(4-trifluoromethyl-phenyl)-urea; yield 45%.

¹H NMR (CDCl₃): δ 1.08 (6H, t), 2.61-2.74 (6H, m), 3.45 (6H, s), 6.59 (1H, s), 6.96-7.10 (4H, m), 7.32 (2H, d) 7.46 (2H, d), 7.80 (1H, s), 11.64 (1H, s, broad).

1-(4-Chloro-phenyl)-3-{2-[3-(3-methoxy-phenyl)-1-(2-piperidin-1-yl-ethyl)-thioureido]-ethyl}-thiourea.

¹H NMR (CDCl₃): δ 1.45-2.07 (6H, m), 3.06 (2H, s, broad), 3.47 (2H, s), 3.63 (2H, s, broad), 3.77 (3H, s), 3.87 (2H, d), 3.93 (2H, d), 4.34 (2H, s), 6.79 (1H, d), 7.00 (1H, d), 7.06 (1H, s) 7.23 (1H, t), 7.28-7.39 (4H, m).

1-(2-Piperidin-1-yl-ethyl)-3-(4-trifluoromethyl-phenyl)-1-{2-[3-(3-trifluoromethyl-phenyl)-ureido]-ethyl}-urea; yield 100%, (Compound G).

¹H NMR (CDCl₃) δ 1.54 (2H, s), 1.65 (4H, s), 2.61 (6H, d), 3.47 (6H, d), 6.66 (1H, s), 7.03 (1H, s), 7.11-7.21 (2H, m), 7.38-7.52 (4H, m), 7.63 (1H, s), 8.05 (1H, s, broad) 11.04 (1H, s, broad).

1-{2-[3-(4-Chloro-phenyl)-ureido]-ethyl}-1-(2-piperidin-1-yl-ethyl)-3-(4-trifluoromethyl-phenyl)-urea; yield 51%.

¹H NMR (CDCl₃): δ 1.53 (2H, s) 1.64 (4H, m), 2.68 (6H, s), 3.45 (6H, d), 6.61 (1H, s), 7.01 (4H, d), 7.45 (4H, d), 7.75 (1H, s), 10.97 (1H, s, broad).

1-(4-Chloro-benzyl)-1-{2-[3-(4-chloro-phenyl)-ureido]-ethyl}-3-(4-trifluoromethyl-phenyl)-urea; yield 73%.

¹H NMR (CD₃OD): δ 3.30 (2H, t), 3.51 (2H, t), 4.65 (2H, s), 7.23 (2H, d), 7.29-7.39 (6H, m), 7.52 (2H, d) 7.71 (2H, d).

1-{2-[3-(4-Bromo-phenyl)-1-(2-diethylamino-ethyl)ureido]-ethyl}-3-(2,6-dichloro-pyridin-4-yl)-urea,

(Compound M); yield 21%.

Esi-Ms m/z 547 (M+H⁺)

1-{3-[3-(3-Chloro-phenyl)-ureido]-propyl}-1-(2-dimethylamino-ethyl)-3-(4-trifluoromethyl-phenyl)-urea; yield 87%.

¹H NMR (CDCl₃): δ 1.66-1.79 (2H, m), 2.39 (6H, s), 2.56-2.65 (2H, m), 3.19-3.28 (2H, m), 3.29-3.34 (2H, m), 3.38 (2H, t), 6.44 (1H, t), 6.89 (1H, d), 7.03-7.15 (2H, m), 7.33 (2H, d), 7.40 (1H, s), 7.45 (2H, d), 7.86 (1H, s), 11.47 (1H, s, broad).

1-{2-[1-(2-Diethylamino-ethyl)-3-(4-trifluoromethylphenyl)-ureido]-ethyl}-3-naphthalen-1-yl-urea; yield 69%.

¹H NMR (CDCl₃): δ 1.01 (6H, 2t), 2.52-2.63 (6H, m), 3.28 (2H, d), 3.39-3.49 (4H, m), 6.27 (1H, s), 7.16 (2H, d), 7.25-7.37 (3H, m), 7.42 (2H, t), 7.56-7.71 (3H, m), 7.82 (1H, d), 7.98 (1H, d), 11.25 (1H, s, broad).

1-{2-[3-(4-Bromo-phenyl)-1-(2-diethylamino-ethyl)ureido]-ethyl}-3-naphthalen-1-yl-urea; yield 69%.

¹H NMR (CDCl₃): δ 0.97 (6H, t), 2.43-2.58 (6H, m, broad), 3.22 (2H, s), 3.39 (4H, s), 6.47 (1H, s), 6.96 (2H, d), 7.10 (2H, d), 7.29 (1H, t), 7.35-7.44 (2H, m), 7.64 (2H, t), 7.80 (1H, d), 7.91-7.99 (2H, m), 10.98 (1H, s, broad).

1-{2-[3-(3-Chloro-phenyl)-ureido]-ethyl}-1-(3-diethylamino-propyl)-3-(4-trifluoromethyl-phenyl)-urea; yield 100%.

¹H NMR (CDCl₃): δ 1.05 (6H, t), 1.79-1.90 (2H, m), 2.53 (2H, t), 2.65 (4H, q), 3.46 (6H, s), 6.47 (1H, s, broad), 6.90 (1H, d), 6.96 (1H, s, broad) 7.04 (1H, t), 7.33 (1H, s), 7.41 (2H, d), 7.48 (2H, d), 7.72 (1H, s, broad), 10.32 (1H, s, broad).

1-{2-[3-(4-Bromo-phenyl)-ureido]-ethyl}-1-(3-diethylamino-propyl)-3-(4-trifluoromethyl-phenyl)-urea; yield 100%.

¹H NMR (CDCl₃): δ 1.04 (6H, t), 1.79-1.89 (2H, m), 2.52 (2H, t), 2.65 (4H, q), 3.45 (6H, s), 6.40 (1H, s, broad), 7.06 (2H, d), 7.25 (2H, d), 7.37-7.57 (5H, m), 10.30 (1H, s, broad).

1-(2-Diethylamino-ethyl)-1-{2-[3-(4-diethylamino-phenyl)thioureido]-ethyl}-3-(4-trifluoromethyl-phenyl)-urea; yield 70%.

¹H NMR (CDCl₃): δ 1.08 (6H, t), 1.16 (6H, t), 2.61-2.74 (6H, m), 3.30-3.45 (6H, m), 3.53 (2H, t), 3.80 (2H, q), 6.64 (2H, d), 7.04 (2H, m), 7.22 (2H, d), 7.43 (1H, s), 7.45 (2H, d), 11.26 (1H, s, broad).

3-(4-Chloro-phenyl)-1-(2-pyrrolidin-1-yl-ethyl)-1-{2-[3-(3-trifluoromethyl-phenyl)-ureido]-ethyl}-urea; yield 78%.

¹H NMR (CD₃OD): δ 1.79-1.90 (4H, m), 2.69 (4H, s), 2.80 (2H, t), 3.41 (2H, t), 3.48-3.59 (4H, m), 7.16-7.28 (3H, m), 7.34-7.45 (3H, m), 7.49 (1H, d), 7.91 (1H, s). 1-(4-Chloro-phenyl)-3-{2-[3-(3-chloro-phenyl)-1-(2-piperidin-1-yl-ethyl)-ureido]-ethyl}-urea; yield 25%.

¹H NMR (CDCl₃): δ 1.52 (2H, s), 1.58-1.68 (4H, m), 2.47-2.66 (6H, m), 3.35-3.50 (6H, m), 6.56 (1H, s), 6.96-7.11 (5H, m), 7.16 (2H, d), 7.40 (1H, s), 7.80 (1H, s), 10.76 (1H, s, broad).

1-{2-[3-(3-Chloro-phenyl)-1-(2-piperidin-1-yl-ethyl)ureido]-ethyl}-3-(3-trifluoromethyl-phenyl)-urea, (Compound D); yield 10%.

¹H NMR (CDCl₃): δ 1.51 (2H, s), 1.56-1.66 (4H, m), 2.45-2.64 (6H, m), 3.37-3.50 (6H, m), 6.55 (1H, s), 6.91-6.97 (1H, m), 7.06-7.24 (5H, m), 7.38 (1H, s), 7.62 (1H, s), 7.99 (1H, broad), 10.89 (1H, broad).

1-(2-Dimethylamino-ethyl)-3-(4-trifluoromethyl-phenyl)-1-{2-[3-(3-trifluoromethyl-phenyl)-ureido]-ethyl}-urea; yield 59%.

¹H NMR (CDCl₃): δ 2.42 (6H, s), 2.68 (2H, d), 3.41-3.53 (6H, m), 6.51 (1H, s), 7.13-7.23 (3H, m), 7.32 (2H, d), 7.46 (2H, d), 7.62 (1H, s), 7.87 (1H, s, broad), 11.74 (1H, s, broad).

1-(4-Chloro-phenyl)-3-{2-[3-(3-methoxy-phenyl)-1-(2-piperidin-1-yl-ethyl)-ureido]-ethyl}-urea; recrystallized from CHCl₃:hexane; yield 63%.

¹H NMR (CDCl₃): δ 1.52 (2H, s), 1.61-1.70 (4H, m), 2.50-2.64 (6H, m), 3.41 (4H, s), 3.46-3.52 (2H, m), 3.70 (3H, s), 6.58 (1H, d), 6.79 (1H, s), 6.85-6.96 (3H, m), 7.03 (2H, d), 7.17 (1H, t), 7.74 (1H, s), 10.65 (1H, s, broad).

1-{2-[3-(3-Methoxy-phenyl)-1-(2-piperidin-1-yl-ethyl)ureido]-ethyl}-3-(3-trifluoromethyl-phenyl)-urea; yield 58%.

¹H NMR (CDCl₃): δ 1.48 (2H, s), 1.57-1.66 (4H, m), 2.54-2.62 (6H, m), 3.36-3.49 (6H, m), 3.71 (3H, s), 6.47 (1H, s), 6.56 (1H, d), 6.89 (1H, d), 6.94 (1H, s), 7.16 (2H, t), 7.24 (2H, d), 7.70 (1H, s), 8.39 (1H, d), 10.52 (1H, broad).

1-{2-[1-(2-Pyrrolidin-1-yl-ethyl)-3-(4-trifluoromethylphenyl)-ureido]-ethyl}-3-(3-trifluoromethyl-phenyl)-urea, (Compound H); yield 68%.

¹H NMR (CDCl₃): δ 1.85 (4H, s), 2.59 (2H, s, broad), 2.90-2.97 (2H, m), 3.30-3.49 (6H, m), 3.61 (2H, s, broad), 7.25 (1H, d), 7.38 (1H, t), 7.49 (3H, d), 7.61 (1H, s), 7.84 (2H, d), 8.28 (1H, s), 9.35 (1H, s, broad), 10.13 (1H, s, broad).

1-(4-Chloro-phenyl)-3-{2-[1-(2-pyrrolidin-1-yl-ethyl)-3-(4-trifluoromethyl-phenyl)-ureido]-ethyl}-urea; yield 84%.

¹H NMR (CDCl₃): δ 1.80-1.94 (4H, m), 2.74 (4H, s), 2.84 (2H, d), 3.40-3.52 (6H, m), 6.30 (1H, s), 7.07-7.16 (4H, m), 7.22 (1H, broad), 7.32 (2H, d), 7.50 (2H, d), 11.41 (1H, broad).

1-(4-Methoxy-phenyl)-3-{2-[1-(2-pyrrolidin-1-yl-ethyl)-3-(4-trifluoromethyl-phenyl)-ureido]-ethyl}-urea; yield 52%.

¹H NMR (CDCl₃): δ 1.87 (4H, s), 2.69 (4H, s), 2.79 (2H, s), 3.44 (6H, s), 3.74 (3H, s), 6.21 (1H, s), 6.74 (2H, d), 7.06 (2H, d), 7.19 (1H, s), 7.31 (2H, d), 7.45 (2H, d), 11.21 (1H, s, broad).

1-(4-Bromo-phenyl)-3-{3-[1-(2-pyrrolidin-1-yl-ethyl)-3-(4-trifluoromethyl-phenyl)-thioureido]-propyl}-urea; yield 65%.

¹H NMR (CD₃OD): δ 1.77-1.93 (6H, m), 2.71-2.80 (4H, m), 2.86 (2H, t), 3.25 (2H, t), 3.46 (2H, t), 3.52 (2H, t), 7.27-7.38 (4H, m), 7.46-7.56 (4H, m).

1-(3-Chloro-phenyl)-3-{3-[1-(2-pyrrolidin-1-yl-ethyl)-3-(4-trifluoromethyl-phenyl)-ureido]-propyl}-urea; yield 78%.

¹H NMR (CD₃OD): δ 1.80-1.91 (2H, m), 1.96 (4H, s), 2.91-3.11 (6H, m), 3.27 (2H, t), 3.49 (2H, t), 3.61 (2H, t), 6.95 (1H, d), 7.14-7.23 (2H, m), 7.51-7.60 (4H, m), 7.90 (1H, s).

3-(4-Chloro-phenyl)-1-(2-pyrrolidin-1-yl-ethyl)-1-{2-[3-(4-trifluoromethyl-phenyl)-ureido]-ethyl}-urea; yield 70%.

¹H NMR (CD₃OD) δ 1.78-1.90 (4H, m), 2.69 (4H, s), 2.80 (2H, t), 3.41 (2H, t), 3.43-3.56 (4H, m), 7.21 (2H, d), 7.35 (2H, d), 7.50-7.60 (4H, m).

1-(3-Chloro-phenyl)-3-{2-[1-(3-pyrrolidin-1-yl-propyl)-3-(4-trifluoromethyl-phenyl)-ureido]-ethyl}-urea; yield 94%.

¹H NMR (CDCl₃): δ 1.75-1.91 (6H, m), 2.57 (6H, s), 3.40-3.53 (6H, m), 6.49 (1H, s), 6.89 (1H, d), 6.95 (1H, s), 7.04 (1H, t), 7.35 (1H, s), 7.40 (2H, d), 7.47 (2H, d), 7.76 (1H, s, broad), 10.28 (1H, s).

1-(4-Bromo-phenyl)-3-{2-[1-(3-pyrrolidin-1-yl-propyl)-3-(4-trifluoromethyl-phenyl)-ureido]-ethyl}-urea; yield 43%.

¹H NMR (CDCl₃): δ 1.77-1.94 (6H, m), 2.52-2.64 (6H, m), 3.43-3.53 (6H, m), 6.18 (1H, s, broad), 7.15 (3H, d), 7.30 (2H, d), 7.41 (2H, d), 7.51 (2H, d), 10.25 (1H, s).

1-{2-[3-(4-Chloro-phenyl)-ureido]-ethyl}-1-(3-dimethylamino-propyl)-3-(4-trifluoromethyl-phenyl)-urea.

¹H NMR (CD₃OD): δ 1.75-1.86 (2H, m), 2.27 (6H, s), 2.36 (2H, t), 3.38-3.51 (6H, m), 7.20 (2H, d), 7.35 (2H, d), 7.51 (2H, d), 7.60 (2H, d).

1-(3-Dimethylamino-propyl)-1-[2-(3-phenyl-ureido)-ethyl]-3-(4-trifluoromethyl-phenyl)-urea.

¹H NMR (CD₃OD): δ 1.75-1.85 (2H, m), 2.27 (6H, s), 2.37 (2H, t), 3.40-3.55 (6H, m), 6.98 (1H, t), 7.24 (2H, t), 7.35 (2H, d), 7.51 (2H, d), 7.61 (2H, d).

1-{2-[3-(4-Chloro-phenyl)-ureido]-ethyl}-1-(2-diisopropylamino-ethyl)-3-(4-trifluoromethyl-phenyl)-urea.

¹H NMR (CD₃OD): δ 1.10 (12H, d), 2.77 (2H, t), 3.10-3.21 (2H, m), 3.39-3.53 (6H, m), 7.22 (2H, d), 7.36 (2H, d), 7.46-7.57 (4H, m).

1-{2-[3-(4-Bromo-phenyl)-ureido]-ethyl}-1-(2-diisopropylamino-ethyl)-3-(4-trifluoromethyl-phenyl)-urea.

¹H NMR (CD₃OD): δ 1.09 (12H, d), 2.75 (2H, s), 3.06-3.21 (2H, m), 3.38-3.53 (6H, m), 7.28-7.39 (4H, m), 7.45-7.57 (4H, m).

1-(2-Diisopropylamino-ethyl)-1-[2-(3-phenyl-ureido)ethyl]-3-(4-trifluoromethyl-phenyl)-urea.

¹H NMR (CD₃OD): δ 1.10 (12H, d), 2.77 (2H, t), 3.11-3.22 (2H, m), 3.40-3.54 (6H, m), 6.99 (1H, t), 7.25 (2H, t), 7.36 (2H, d), 7.46-7.56 (4H, m).

1-{2-[1-(2-Dimethylamino-ethyl)-3-(4-trifluoromethylphenyl)-ureido]-ethyl}-3-naphthalen-1-yl-urea.

¹H NMR (CD₃OD): δ 2.28 (6H, s), 2.53 (2H, t), 3.36-3.51 (6H, m), 7.36-7.52 (7H, m), 7.60-7.64 (2H, 2d), 7.82 (1H, d), 7.98 (1H, d).

1-{2-[3-(4-Bromo-phenyl)-ureido]-ethyl}-1-[3-(4-methylpiperazin-1-yl)-propyl]-3-(4-trifluoromethyl-phenyl)-urea.

¹H NMR (CDCl₃): δ 1.74-1.83 (2H, m), 2.25 (3H, s), 2.31-2.58 (10H, m), 3.40 (6H, s), 6.30 (1H, s, broad), 7.06 (2H, d), 7.26 (2H, d), 7.50 (4H, s), 7.66 (1H, s, broad), 9.32 (1H, s).

### EXAMPLE 8

### 1-{2-[1-(2-Pyrrolidin-1-yl-ethyl)-3-(4-trifluoromethylphenyl)-ureido]-ethyl}-3-quinolin-6-yl-urea, (Compound I)

4-Methyl-morpholine (584 mg, 5.8 mmol) was added to a solution of 2,4,6-trichloro-[1,3,5]triazine (355 mg, 1.92 mmol) in CH₂Cl₂ (20 mL) at 0° C. A slurry of quinoline-6-carboxylic acid (1.00 g, 5.8 mmol) in CH₂Cl₂ was added. The reaction mixture was stirred at 0° C. for 4.5 hours. The solution was filtered trough celite and NaN₃ (375 mg, 5.8 mmol) was added to the filtrate. The reaction mixture was allowed to stand at room temperature over night stirring all the time. The solution was extracted first with Na₂CO₃ and then with water. The organic layer was dried over sodium sulphate and evaporated. The crude product was purified by flash silica gel chromatography using, EtOAc:Heptane 1:1 as the eluent. Concentration in vacuum of the product-rich fractions provided quinoline-6-carbonyl azide (310 mg, 28%).

¹H NMR (CDCl₃): δ 7.49 (1H, q), 8.15 (1H, d), 8.26 (2H, d), 8.57 (1H, s), 9.02 (1H, d).

A microwave-assisted reaction were carried out in capped vials using a microwave oven with temperature and pressure control. A solution of quinoline-6-carbonyl azide (29.9 mg, 0.15 mmol) in 1.5 mL CH₂Cl₂ was heated at 110° C. for 15 minutes. 1-(2-Amino-ethyl)-1-(2-pyrrolidin-1-yl-ethyl)-3-(4-trifluoromethyl-phenyl)-urea (52 mg 0.15 mmol) was added and the reaction mixture was stirred for 1 hour. The product was purified by flash silica gel chromatography using, MeOH: Et₃N 100:1 as the eluent. Concentration in vacuum of the product-rich fractions provided the title compound (45 mg, 55%).

¹H NMR (CDCl₃): δ 1.90 (4H, s), 2.75 (4H, s), 2.87 (2H, d), 3.52 (6H, s), 6.52 (1H, s, broad) 7.22-7.32 (2H, m), 7.35 (2H, d), 7.48 (2H, d), 7.72 (1H, s), 7.85 (1H, d), 7.94 (2H, d), 8.75 (1H, d), 11.42 (1H, s)

Other “symmetric” diurea derivatives of the formula (Ia) were prepared by the method described in Example 2 are:
1-Benzyl-3-(3-trifluoromethyl-phenyl)-1-{2-[3-(3-trifluoromethyl-phenyl)-thioureido]-ethyl}-thiourea.
3-(2-Chloro-4-trifluoromethyl-phenyl)-1-{2-[3-(2-chloro-4-trifluoromethyl-phenyl)-ureido]-ethyl}-1-(2-diethylamino-ethyl)-urea.
1-{2-[1-(2-Pyrrolidin-1-yl-ethyl)-3-(3-trifluoromethylphenyl)-thioureido]-ethyl}-3-(3-trifluoromethyl-phenyl)thiourea.
1-(4-Chloro-phenyl)-3-{2-[3-(4-chloro-phenyl)-1-(2-dimethylamino-ethyl)-thioureido]-ethyl}-thiourea.
1-(4-Chloro-benzyl)-3-(4-trifluoromethyl-phenyl)-1-{2-[3-(4-trifluoromethyl-phenyl)-ureido]-ethyl}-urea.
1-{2-[1-(4-Methyl-benzyl)-3-(4-trifluoromethyl-phenyl)ureido]-ethyl}-3-(4-trifluoromethyl-phenyl)-urea.
1-(2-Dimethylamino-ethyl)-3-(4-trifluoromethyl-phenyl)-1-{2-[3-(4-trifluoromethyl-phenyl)-ureido]-ethyl}-urea.
1-(3-Diethylamino-propyl)-3-(4-trifluoromethyl-phenyl)-1-{2-[3-(4-trifluoromethyl-phenyl)-ureido]-ethyl}-urea.
1-[3-(4-Methyl-piperazin-1-yl)-propyl]-3-(4-trifluoromethyl-phenyl)-1-{2-[3-(4-trifluoromethyl-phenyl)ureido]-ethyl}-urea.
1-{2-[1-Benzyl-3-(3-trifluoromethyl-phenyl)-ureido]-ethyl}-3-(3-trifluoromethyl-phenyl)-urea.
1-{2-[1-Benzyl-3-(4-trifluoromethyl-phenyl)-ureido]-ethyl}-3-(4-trifluoromethyl-phenyl)-urea.
1-{2-[1-(2-Diethylamino-ethyl)-3-(3-trifluoromethylphenyl)-thioureido]-ethyl}-3-(3-trifluoromethyl-phenyl)thiourea.
3-(3-Fluoro-phenyl)-1-{2-[3-(3-fluoro-phenyl)-ureido]ethyl}-1-(2-piperidin-1-yl-ethyl)-urea.
1-{2-[1-(2-Fluoro-benzyl)-3-(4-trifluoromethyl-phenyl)ureido]-ethyl}-3-(4-trifluoromethyl-phenyl)-urea.
1-{3-[1-(2-Pyrrolidin-1-yl-ethyl)-3-(4-trifluoromethylphenyl)-ureido]-propyl}-3-(4-trifluoromethyl-phenyl)-urea.
1-(3-Dimethylamino-propyl)-3-(4-trifluoromethyl-phenyl)-1-{3-[3-(4-trifluoromethyl-phenyl)-ureido]-propyl}-urea.

Other “asymmetric” diurea derivatives of the formula (Ib), prepared by the method described in Example 7, are:
1-{2-[3-(4-Chloro-phenyl)-1-(2-diethylamino-ethyl)thioureido]-ethyl}-3-(3-trifluoromethyl-phenyl)-urea.
1-(2-Dimethylamino-ethyl)-1-{2-[3-(4-methoxy-phenyl)-ureido]-ethyl}-3-(4-trifluoromethyl-phenyl)-urea.
1-(3-Dimethylamino-propyl)-1-{2-[3-(4-methoxy-phenyl)ureido]-ethyl}-3-(4-trifluoromethyl-phenyl)-urea.
1-(2-Diethylamino-ethyl)-1-{2-[3-(4-methoxy-phenyl)ureido]-ethyl}-3-(4-trifluoromethyl-phenyl)-urea.
1-[2-(3-(4-Chloro-phenyl)-thioureido)-ethyl]-1-(2-diethylamino-ethyl)-3-(3-methoxy-phenyl)-thiourea.
1-[2-(3-Phenyl-ureido)-ethyl]-1-(2-piperidin-1-yl-ethyl)-3-(4-trifluoromethyl-phenyl)-urea.
1-{2-[3-(4-Methoxy-phenyl)-ureido]-ethyl}-1-(2-piperidin-1-yl-ethyl)-3-(4-trifluoromethyl-phenyl)-urea.
1-[2-(3-Phenyl-ureido)-ethyl]-1-(2-pyrrolidin-1-ylethyl)-3-(4-trifluoromethyl-phenyl)-urea.
1-{2-[3-(4-Bromo-phenyl)-ureido]-ethyl}-1-(4-chlorobenzyl)-3-(4-trifluoromethyl-phenyl)-urea.
1-(2-Diisopropylamino-ethyl)-1-{2-[3-(4-methoxy-phenyl)ureido]-ethyl}-3-(4-trifluoromethyl-phenyl)-urea.
1-{3-[3-(3-Chloro-phenyl)-ureido]-propyl}-1-(3-dimethylamino-propyl)-3-(4-trifluoromethyl-phenyl)-urea.
1-{2-[3-(4-Bromo-phenyl)-1-(2-dimethylamino-ethyl)ureido]-ethyl}-3-naphthalen-1-yl-urea.
1-{2-[3-(4-Bromo-phenyl)-1-(2-dimethylamino-ethyl)ureido]-ethyl}-3-naphthalen-1-yl-urea.
1-{2-[3-(4-Diethylamino-phenyl)-1-(2-dimethylaminoethyl)-thioureido]-ethyl}-3-(4-trifluoromethyl-phenyl)-urea.
1-{2-[1-(2-Diethylamino-ethyl)-3-(4-diethylamino-phenyl)thioureido]-ethyl}-3-(4-trifluoromethyl-phenyl)-urea.
1-(4-Chloro-phenyl)-3-{2-[1-(3-dimethylamino-propyl)-3-(3-methoxy-phenyl)-thioureido]-ethyl}-thiourea.
1-(2-Diethylamino-ethyl)-3-(3-methoxy-phenyl)-1-(2-[3-(3-trifluoromethyl-phenyl)-thioureido]-ethyl}-thiourea.
1-{2-[3-(4-Chloro-phenyl)-thioureido]-ethyl}-3-(3-methoxy-phenyl)-1-(2-pyrrolidin-1-yl-ethyl)-thiourea.
1-(4-Bromo-phenyl)-3-{2-[1-(4-methyl-benzyl)-3-(4-trifluoromethyl-phenyl)-ureido]-ethyl}-urea.
1-(3-Chloro-phenyl)-3-{2-[1-(4-methyl-benzyl)-3-(4-trifluoromethyl-phenyl)-ureido]-ethyl}-urea.
1-{3-[3-(4-Bromo-phenyl)-ureido]-propyl}-1-(2-dimethylamino-ethyl)-3-(4-trifluoromethyl-phenyl)-urea.
1-{3-[3-(4-Bromo-phenyl)-ureido]-propyl}-1-(3-dimethylamino-propyl)-3-(4-trifluoromethyl-phenyl)-urea.
1-{2-[1-(2-Diethylamino-ethyl)-3-(4-trifluoromethylphenyl)-ureido]-ethyl}-3-naphthalen-1-yl-urea.
1-{2-[3-(4-Bromo-phenyl)-1-(2-pyrrolidin-1-yl-ethyl)ureido]-ethyl}-3-(2,6-dichloro-pyridin-4-yl)-urea.
1-{2-[3-(3-Chloro-phenyl)-ureido]-ethyl}-1-[3-(4-methylpiperazin-1-yl)-propyl]-3-(4-trifluoromethyl-phenyl)-urea.
1-{2-[3-(4-Diethylamino-phenyl)-thioureido]-ethyl}-1-(2-pyrrolidin-1-yl-ethyl)-3-(4-trifluoromethyl-phenyl)-urea.
1-{2-[3-(4-Diethylamino-phenyl)-1-(2-dimethylaminoethyl)-thioureido]-ethyl}-3-(3-trifluoromethyl-phenyl)-urea.
1-{2-[3-(4-Diethylamino-phenyl)-1-(2-pyrrolidin-1-ylethyl)-thioureido]-ethyl}-3-(4-trifluoromethyl-phenyl)-urea.
1-(2-Diethylamino-ethyl)-1-{2-[3-(4-diethylamino-phenyl)ureido]-ethyl}-3-(4-trifluoromethyl-phenyl)-urea.
1-{2-[3-(4-Diethylamino-phenyl)-1-(2-dimethylaminoethyl)-ureido]-ethyl}-3-(4-trifluoromethyl-phenyl)-urea.
1-(3-Diethylamino-propyl)-1-[2-(3-phenyl-ureido)-ethyl]-3-(4-trifluoromethyl-phenyl)-urea.

### Pharmacological Methods

The compounds of formula (I) were assayed for inhibition of IL-2 production.

### Inhibition of IL-2 Production

The compounds to be evaluated were dissolved in DMSO and the dilution series of the compounds were prepared in DMSO. The series were further diluted in cell culture medium (RPMI 1640 with ultraglutamine, 10% foetal calf serum (FCS)) to obtain a final assay concentration of DMSO of 0.1% in 200 μl total volume. The compounds were plated (2 μl/well) on opaque white assay plates together with controls.

Peripheral blood mononuclear cells (PBMC) were isolated from human blood drawn from healthy volunteers, by density gradient separation over Ficoll-Paque. T-cells (CD4+) were obtained with positive selection using magnetic cell sorting (MACS). The cells were resuspended at 1×10⁶ cells/ml in cell culture medium (RPMI 1640 with ultraglutamine, 10% FCS, 10 mM hepes, 1 mM sodium pyruvate and 0.1 mg/ml gentamicin).

The cells (1×10⁵/well) were added to the assay plates containing the diluted compounds and pre-incubated for 30 min at 37° C. in a humidified atmosphere of 5% carbon dioxide. The cells were stimulated with 10 ng/ml phorbol myristate acetate (PMA) and 250 ng/ml ionomycin and the plates were incubated for 4 hours at 37° C. in a humidified atmosphere of 5% carbon dioxide. Approximately 100 μl of the supernatants were removed and transferred to a separate microtiter plate and the remaining cells were lysed (Nucleotide Releasing Reagent, ViaLight™, Cambrex). All the plates were kept at −20° C. pending analysis. Human interleukin-2 (IL-2) was analysed with a standard ELISA kit (OptEIA™, Pharmingen) according to the manufacturer's instructions. Viability was assessed by measuring adenosine triphosphate (ATP) content by adding luciferase (ATP monitoring reagent, ViaLight™, Cambrex) to the lysed cells and measuring luminescence, all according to the manufacturer's specifications.

The % effect of each concentration of compound was calculated compared to non-treated stimulated cells. Non-linear regression, a modified Hill-plot (y=(a−d)/(1+(x/c)\hat{b})+d) was used to calculate the concentration for y=50% (IC₅₀).

### Inhibition of Other Cytokines

By similar methods using peripheral blood mononuclear cells, appropriate stimuli, and commercially available ELISA kits, for a particular cytokine, inhibition of IL-6, TNF-α and IFN-γ were demonstrated.

### Induction of Apoptosis

The induction of apoptosis can be observed by measuring Annexin V-binding to cells (Van Engeland et al. 1998). Primary human CD4⁺ T cells were isolated from peripheral blood from healthy volunteers as described above. Cells were cultured immediately after purification at a density of 2×10⁶ cells/ml in RPMI 1640 medium supplemented with 10% FCS, Gentamycin (100 μg/ml), Hepes (10 mM) and Sodium Pyruvate (1 mM). Cells were stained with annexin V-FITC and propidium iodide by using the ApoAlert Annexin V-FITC Apoptosis Kit (Clontech) according to manufacturer's instructions. Flow cytometry analysis was performed using a FACScan (Becton Dickinson).

Alternatively, induction of apoptosis can be demonstrated measuring cleavage of the caspase-substrate PARP (poly(ADP-ribose)polymerase) (Tang et al. 1996). Cell lysates were prepared by lysing 2×10⁶ PBS-washed cells in 50 μl buffer containing 20 mM Tris-HCl, pH 7.7, 250 mM NaCl, 3 mM EDTA, 3 mM EGTA 0.5% NP-40 supplemented with 1 mM p-nitrophenyl phosphate (PNPP), 10 mM β-glycerophosphate, 100 μM Na-vanadate and 1 mM phenylmethansulfonyl fluoride (PMSF). The protein concentrations were determined by using Bio-Rads protein assay and thereafter equal amounts of protein was loaded onto precasted NuPAGE™ Tris-Bis gels (Novex). After electrophoresis, the proteins were transferred to nitrocellulose membrane and probed with a polyclonal rabbit antibody directed against PARP (Roche). Proteins were visualised after incubations with a horseradish peroxidase-conjugated secondary anti-body and ECL reagents (Amersham Bioscience).

Another method for measuring apoptosis involves ylsualising specific DNA fragmentation (Willingham et al. 1999). DNA was extracted using Suicide-Track DNA Ladder Isolation Kit (Oncogene Research Products) according to manufacturer's instructions. DNA fragmentation was visualised on 1.5% agarose gels in the presence of ethidium bromide.

### Summary of Test Results

Among preferred compounds is 1-(2-diethylaminoethyl)-3-(3-trifluoromethyl-phenyl)-1-{2-[3-(3-trifluoromethyl-phenyl)-ureido]-ethyl}-urea, hydrochloride hereinafter called Compound A.

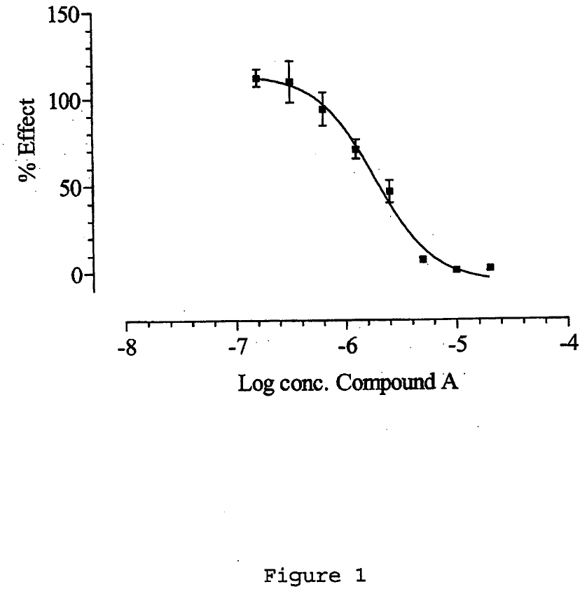

The effect of Compound A on PMA/Ionomycin stimulated IL-2 production in human T-cells was determined (FIG. 1). The IC₅₀ of Compound A was 2±1 μM (mean±S.D., n=30).

Examples of other compounds showing similar effects on IL-2 production are shown below in table 1 (including the result for Compound A).

TABLE 1
Inhibition of PMA/Ionomycin stimulated IL-2 production
in human T-cells for compounds A-M (mean, n = 2).

| Compound | IC50 IL-2 μM |
|---|---|
| A | 2 |
| B | 2 |
| C | 2 |
| D | 2 |
| E | 1 |
| F | 3 |
| G | 1 |
| H | 1 |
| I | 5 |
| J | 3 |
| K | 2 |
| L | 2 |
| M | 2 |

The effect of Compound A on PMA/Ionomycin stimulated IL-6, TNF-α and IFN-γ production in human peripheral blood mononuclear cells was determined. The IC50 values of Compound A were 3±1 μM (mean±S.D., n=3) for IL-6, 3±1 μM (mean±S.D., n=3) for TNF-α and 4±1 μM (mean±S.D., n=3) for IFN-γ.

The effect of Compound A on apoptosis induction in human T-cells was determined by methods mentioned above. Significant apoptosis induction was observed at >4 μM of Compound A.

Effective quantities of the compounds of formula (I) are preferably administered to a patient in need of such treatment according to usual routes of administration and formulated in usual pharmaceutical compositions comprising an effective amount of the active ingredient and one or more suitable pharmaceutically acceptable excipients or carriers. Such compositions may take a variety of forms, e.g. solutions, suspensions, emulsions, tablets, capsules, and powders prepared for oral administration, aerosols for inhalation, sterile solutions for parental administration, suppositories for rectal administration or suitable topical formulations. Conventional procedures for the selection and preparation of suitable pharmaceutical formulations are described, for example, in “Pharmaceuticals—The Science of Dosage Form Design”, M. B. Aulton, Churchill Livingstone, (1988).

A suitable daily dose for use in the treatment of rheumatoid arthritis is contemplated to vary between 0.0005 mg/kg to about 10 mg/kg body weight, in particular between 0.005 mg/kg to 1 mg/kg body weight, depending upon the specific condition to be treated, the age and weight of the specific patient, and the specific patient's response to the medication. The exact individual dosage, as well as the daily dosage, will be determined according to standard medical principles under the direction of a physician.

Various additives to enhance the stability or ease of administration of the drug are contemplated. The pharmaceutical composition may also contain additional therapeutically useful substances other than a compound of formula (I).

### REFERENCES

Abbas, A. K., Murphy, K. M., and Sher, A. (1996) Functional diversity of helper T lymphocytes. Nature 383:787-793.
Aulton, M. E. Parmaceutics. 1988. The Science of Dosage Form Design. Churchill Livingstone, 678 pp. ISBN: 0443055173
Bleesing, J. J. (2003) Autoimmune lymphoproliferative syndrome (ALPS). Curr Pharm Des 9:265-278.
Eguchi, K. (2001) Apoptosis in autoimmune diseases. Intern Med 40:275-284.
Sacca, R., Cuff, C. A., and Ruddle, N. H. (1997) Mediators of inflammation. Curr Opin Immunol 9:851-857.
Tang, D. G., and Porter, A. T. (1996) Apoptosis: A Current Molecular Analysis. Pathol Oncol Res 2:117-131.
Van Engeland, M., Nieland, L. J., Ramaekers, F. C., Schutte, B., and Reutelingsperger, C. P. (1998) Annexin Vaffinity assay: a review on an apoptosis detection system based on phosphatidylserine exposure. Cytometry 31:1-9.
Waldmann, T. A. (1993) The IL-2/IL-2 receptor system: a target for rational immune intervention. Immunol Today 14:264-270.
Waldmann, T. A., Dubois, S., and Tagaya, Y. (2001) Contrasting roles of IL-2 and IL-15 in the life and death of lymphocytes: implications for immunotherapy. Immunity 14:105-110.
Willingham, M. C. (1999) Cytochemical methods for the detection of apoptosis. J Histochem Cytochem 47:1101-1109.
White, M. K., and McCubrey, J. A. (2001) Suppression of apoptosis: role in cell growth and neoplasia. Leukemia. 15:1011-1021.

1. A compound of the general formula I 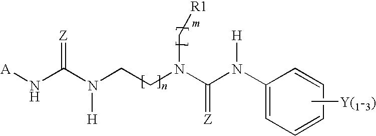
wherein
A is Ph-Y₍₁₋₃₎ or Ar—X₍₀₋₂₎;
R1 is selected from dimethylamino, diethylamino, di-isopropylamino, pyrrolidino, piperidino, and 4-methyl-piperazino;
Ar is selected from phenyl, 1-naphtyl, 2-naphtyl, 2-pyridyl, 3-pyridyl, 4-pyridyl, 6-quinolinyl, and 5-pyrimidinyl;
X₍₀₋₂₎ represents 0 to 2 substituents selected from C1-C6 branched or unbranched alkyls, C1-C6 branched or unbranched alkyloxy, C1-C6 branched or unbranched acyls, fluoro, chloro, bromo, trifluoromethyl, dimethylamino, diethylamino and trifluoromethoxy;
Y₍₁₋₃₎ represents 1 to 3 substituents selected from fluoro, chloro, bromo, dimethylamino, diethylamino, trifluoromethyl, and methoxy;
Z is O or S;
n is 1-3; and
m is 2-4, or
pharmaceutically acceptable salts of the compounds of the general formula I.

2. A compound according to claim 1 having the general formula Ia 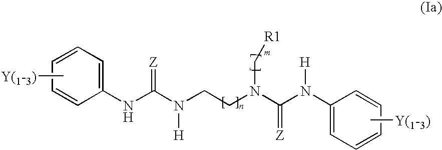
wherein
R1 is selected from dimethylamino, diethylamino, di-isopropylamino, pyrrolidino, piperidino, and 4-methyl-piperazino;
Y₍₁₋₃₎ represents 1 to 3 substituents selected from fluoro, chloro, bromo, dimethylamino, diethylamino, trifluoromethyl, and methoxy;
Z is O or S;
n is 1-3; and
m is 2-4, or
pharmaceutically acceptable salts of the compounds of the general formula Ia.

3. A compound according to claim 1 having the general formula Ib 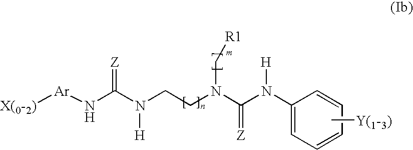
wherein
R1 is selected from dimethylamino, diethylamino, di-isopropylamino, pyrrolidino, piperidino, and 4-methyl-piperazino;
Ar is selected from phenyl, 1-naphtyl, 2-naphtyl, 2-pyridyl, 3-pyridyl, 4-pyridyl, 6-quinolinyl, and 5-pyrimidinyl;
X₍₀₋₂₎ represents 0 to 2 substituents selected from C1-C6 branched or unbranched alkyls, C1-C6 branched or unbranched alkyloxy, C1-C6 branched or unbranched acyls, fluoro, chloro, bromo, trifluoromethyl, dimethylamino, diethylamino and trifluoromethoxy;
Y₍₁₋₃₎ represents 1 to 3 substituents selected from fluoro, chloro, bromo, dimethylamino, diethylamino, trifluoromethyl, and methoxy;
Z is O or S;
n is 1-3; and
m is 24, or
pharmaceutically acceptable salts of the compounds of the general formula Ib.

4. A compound according to claim 1, wherein
R1 is selected from dimethylamino, diethylamino, diisopropylamino, pyrrolidino, piperidino, 4-methyl-piperazino;
n is selected from 1 and 2;
m is selected from 2 and 3;
Y₍₁₋₃₎ is one substituent selected from fluoro, chloro, bromo, trifluoromethyl, dimethylamino and diethylamino.

5. A compound according to claim 1, wherein
Ar is selected from phenyl, 2-naphtyl and 4-pyridyl,
n is selected from 1 and 2;
m is selected from 2 and 3;
Y₍₁₋₃₎ is one of the substituents selected from fluoro, chloro, bromo, and trifluoromethyl.

6. A compound according to claim 1 chosen from the group comprising
1-(2-Diethylamino-ethyl)-3-(3-trifluoromethyl-phenyl)-1-{2-[3-(3-trifluoromethylphenyl)-ureido]-ethyl}-urea;
1-(2-Diethylamino-ethyl)-3-(4-trifluoromethyl-phenyl)-1-{2-[3-(3-trifluoromethylphenyl)-ureido]-ethyl}-urea;
1-(2-Pyrrolidin-1-yl-ethyl)-3-(4-trifluoromethyl-phenyl)-1-{2-[3-(4-trifluoromethylphenyl)-ureido]-ethyl}-urea;
3-(4-Chloro-phenyl)-1-{2-[3-(4-chloro-phenyl)-ureido]-ethyl}-1-(2-pyrrolidin-1-ylethyl)-urea;
1-{2-[3-(3-Chloro-phenyl)-1-(2-piperidin-1-yl-ethyl)-ureido]-ethyl}-3-(3-trifluoromethyl-phenyl)-urea;
1-{2-[3-(4-Chloro-phenyl)-ureido]-ethyl}-1-(2-dimethylamino-ethyl)-3-(4-trifluoromethyl-phenyl)-urea;
3-(4-Bromo-phenyl)-1-{2-[3-(4-bromo-phenyl)-ureido]-ethyl}-1-(2-dimethylamino-ethyl)-urea;
1-(2-Diethylamino-ethyl)-1-[2-(3-phenyl-ureido)-ethyl]-3-(4-trifluoromethylphenyl)-urea;
1-(2-Piperidin-1-yl-ethyl)-3-(3-trifluoromethyl-phenyl)-1-{2-[3-(3-trifluoromethylphenyl)-ureido]-ethyl}-urea;
1-(2-Piperidin-1-yl-ethyl)-3-(4-trifluoromethyl-phenyl)-1-{2-[3-(3-trifluoromethylphenyl)-ureido]-ethyl}-urea;
1-{2-[1-(2-Pyrrolidin-1-yl-ethyl)-3-(4-trifluoromethyl-phenyl)-ureido]-ethyl}-3-(3-trifluoromethyl-phenyl)-urea;
1-{2-[3-(4-Bromo-phenyl)-1-(2-diethylamino-ethyl)-ureido]-ethyl}-3-(2,6-dichloropyridin-4-yl)-urea;
3-(4-Chloro-phenyl)-1-{2-[3-(4-chloro-phenyl)-ureido]-ethyl}-1-(2-diethylaminoethyl)-urea;
1-(2-Dimethylamino-ethyl)-3-(4-trifluoromethyl-phenyl)-1-{2-[3-(3-trifluoromethyl-phenyl)-ureido]-ethyl}-urea;
1-(2-Diethylamino-ethyl)-3-(3-fluoro-phenyl)-1-{2-[3-(3-fluoro-phenyl)-ureido]-ethyl}-urea;
1-{2-[1-(3-Pyrrolidin-1-yl-propyl)-3-(4-trifluoromethyl-phenyl)-ureido]-ethyl}-3-(4-trifluoromethyl-phenyl)-urea;
1-{2-[3-(4-Chloro-phenyl)-ureido]-ethyl}-1-(2-diethylamino-ethyl)-3-(4-trifluoromethyl-phenyl)-urea;
1-{2-[3-(4-Chloro-phenyl)-ureido]-ethyl}-1-(2-diisopropylamino-ethyl)-3-(4-trifluoromethyl-phenyl)-urea;
1-{2-[3-(4-Chloro-phenyl)-ureido]-ethyl}-1-(2-piperidin-1-yl-ethyl)-3-(4-trifluoromethyl-phenyl)-urea;
1-(4-Chloro-phenyl)-3-{2-[3-(4-chloro-phenyl)-1-(2-diethylamino-ethyl)thioureido]-ethyl}-thiourea;
1-{2-[3-(4-Bromo-phenyl)-ureido]-ethyl}-1-(2-diisopropylamino-ethyl)-3-(4-trifluoromethyl-phenyl)-urea;
1-(4-Chloro-phenyl)-3-{2-[1-(2-pyrrolidin-1-yl-ethyl)-3-(4-trifluoromethyl-phenyl)ureido]-ethyl}-urea;
1-{2-[3-(4-Bromo-phenyl)-ureido]-ethyl}-1-(3-diethylamino-propyl)-3-(4-trifluoromethyl-phenyl)-urea;
1-(2-Dimethylamino-ethyl)-1-[2-(3-phenyl-ureido)-ethyl]-3-(4-trifluoromethylphenyl)-urea;
1-(2-Diethylamino-ethyl)-3-(4-trifluoromethyl-phenyl)-1-{2-[3-(4-trifluoromethylphenyl)-ureido]-ethyl}-urea;
1-(4-Bromo-phenyl)-3-{3-[1-(2-pyrrolidin-1-yl-ethyl)-3-(4-trifluoromethyl-phenyl)thioureido]-propyl}-urea;
1-(2-Diisopropylamino-ethyl)-1-[2-(3-phenyl-ureido)-ethyl]-3-(4-trifluoromethylphenyl)-urea;
3-(4-Chloro-phenyl)-1-(2-pyrrolidin-1-yl-ethyl)-1-{2-[3-(3-trifluoromethyl-phenyl)ureido]-ethyl}-urea;
1-(4-Chloro-phenyl)-3-{2-[3-(3-methoxy-phenyl)-1-(2-piperidin-1-yl-ethyl)thioureido]-ethyl}-thiourea;
3-(4-Chloro-phenyl)-1-(2-pyrrolidin-1-yl-ethyl)-1-{2-[3-(4-trifluoromethyl-phenyl)ureido]-ethyl}-urea;
1-{2-[3-(3-Chloro-phenyl)-ureido]-ethyl}-1-(3-diethylamino-propyl)-3-(4-trifluoromethyl-phenyl)-urea; and
1-(2-Diisopropylamino-ethyl)-3-(4-trifluoromethyl-phenyl)-1-{2-[3-(4-trifluoromethyl-phenyl)-ureido]-ethyl}-urea.

7-10. (canceled)

11. A pharmaceutical composition comprising a compound according to claim 1, admixed with one or more pharmaceutically acceptable excipients or carriers.

12. A pharmaceutical composition according to claim 11, wherein the excipients are chosen from the group comprising filling agents, lubricants, flavours, colourings, sweetenings, buffers, acidifying agents, diluents and preservatives.

13. A pharmaceutical composition according to claim 10, which is administered orally, intramuscularly, intravenously, intraperitoneally or subcutaneously, via implants, rectally, intranasally, transdermally, topically, or parenterally.

14. A method of treatment comprising administration of a pharmaceutically effective amount of compound according to claim 1 or a pharmaceutical composition or a pharmaceutical composition comprising said compound with one or more pharmaceutically acceptable excipients or carriers to a subject suffering from an immune disorder which benefit from inhibition of production of IL-2 and other pro-inflammatory cytokines and/or induction of apoptosis in activated T-cells.

15. A method of treatment according to claim 14, wherein the immune disorder are chosen from the group comprising inflammatory diseases, autoimmune diseases, organ and bone marrow transplant rejection and other disorders associated with pro-inflammatory cytokines, especially IL-2, mediated immune response and defective cell regulation.

16. A method of treatment according to claim 14, wherein the immune disorders are chosen from the group comprising acute or chronic inflammation, rheumatoid arthritis, multiple sclerosis, type-1 diabetes, inflammatory bowel disease, psoriasis, graft versus host disease and malignant neoplastic disease.

17. A compound according to claim 2, wherein
R1 is selected from dimethylamino, diethylamino, diisopropylamino, pyrrolidino, piperidino, 4-methyl-piperazino;
n is selected from 1 and 2;
m is selected from 2 and 3;
Y₍₁₋₃₎ is one substituent selected from fluoro, chloro, bromo, trifluoromethyl, dimethylamino and diethylamino.

18. A compound according to claim 3, wherein
R1 is selected from dimethylamino, diethylamino, diisopropylamino, pyrrolidino, piperidino, 4-methyl-piperazino;
n is selected from 1 and 2;
m is selected from 2 and 3;
Y₍₁₋₃₎ is one substituent selected from fluoro, chloro, bromo, trifluoromethyl, dimethylamino and diethylamino.

19. A compound according to claim 3, wherein
Ar is selected from phenyl, 2-naphtyl and 4-pyridyl,
n is selected from 1 and 2;
m is selected from 2 and 3;
Y₍₁₋₃₎ is one of the substituents selected from fluoro, chloro, bromo, and trifluoromethyl.

20. A compound according to claim 4, wherein
Ar is selected from phenyl, 2-naphtyl and 4-pyridyl,
n is selected from 1 and 2;
m is selected from 2 and 3;
Y₍₁₋₃₎ is one of the substituents selected from fluoro, chloro, bromo, and trifluoromethyl.

21. A pharmaceutical composition comprising a compound according to claim 2, admixed with one or more pharmaceutically acceptable excipients or carriers.

22. A pharmaceutical composition comprising a compound according to claim 3, admixed with one or more pharmaceutically acceptable excipients or carriers.

23. A pharmaceutical composition comprising a compound according to claim 4, admixed with one or more pharmaceutically acceptable excipients or carriers.

24. A method of treatment according to claim 15, wherein the immune disorders are chosen from the group comprising acute or chronic inflammation, rheumatoid arthritis, multiple sclerosis, type-1 diabetes, inflammatory bowel disease, psoriasis, graft versus host disease and malignant neoplastic disease.
